(19) United States
(12) Plant Patent Application Publication
(10) Pub. No.: US 2003/0088898 P1
(43) Pub. Date: May 8, 2003

(54) Aerva javancia plant named ' Desert Yearning'

(76) Inventor: Yitzhak Yannai, Moshav Hazeva (IL)

Correspondence Address: Russell D. Orkin, 700 Koppers Building, 436 Sevnth Avenue, Pittsburgh, PA, 15219-1818, US

(21) Appl. No.: 10/007,401

(22) Filed: Nov. 5, 2001

(51) Int. Cl. A01H 5/00

(52) U.S. Cl. PLT/226

(57) ABSTRACT

A new and distinct Aerva javanica plant with a tall stem and light grey-green leaves suitable for cut flower production.

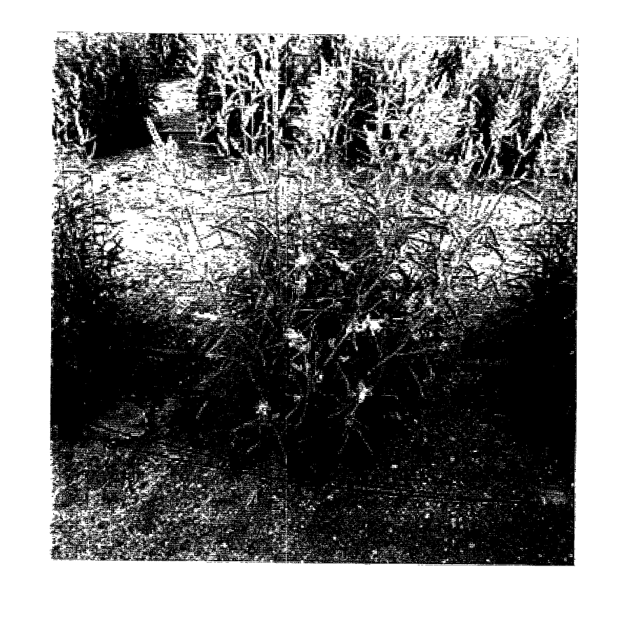

### BACKGROUND OF THE INVENTION

The present invention comprises a new and distinct cultivar of Aerva javanica known by the varietal name ‘Desert Yearning’. The new variety was discovered in the wild in the Arava Valley, Israel at 100 m below sea level. The new variety was selected from a population of Avera javanica plants as having the following desirable characteristics:

1. Hardy and firm stem.

2. Stem length of at least 80 cm.

3. Convenient plant shape for harvesting.

4. Stable flower color.

5. Long shelf life of cut flowers.

6. Attractive, unsymmetrical flowering.

Seeds from the wild variety were collected, germinated and grown to maturity in an ultravoilet area. The new variety was first asexually reproduced by cuttings in June 1997 in Hatzeva, Israel. Continued observations from the vegetative cuttings have confirmed that the distinguishing features of the new variety remain stable and are retained through successive propagations.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new variety. The data which defines these characteristics were collected by asexual reproductions by cuttings carried out in Hatzeva, Israel. The new variety is an ornamental perennial shrub and grows well in sandy soils, open fields, or a greenhouse. Color references are primarily to the R.H.S. Colour Chart of The Royal Horticultural Society of London.

### PLANT

Classification:

Botanical.—Aerva javanica.

Commercial.—Aerva. Height: 15 m. Width: 1.2 m.

Form.—Erect.

Growth.—Vigorous; fast growth rate.

Stem surface.—Smooth texture, dull appearance.

Branches.—Branching: Basal. Surface texture: Smooth; pubescent; no prickles. New growth color: Light grey-green. Mature growth color: Grey-green. Length: Up to 1 mm. Internode length: 60-70 mm. Cross-section: 3 mm in diameter; solid.

Leaves:

Arrangement.—Alternate; simple.

Length.—60-70 mm.

Width.—8-15 mm.

Surface texture.—Soft.

Shape.—Overall: Narrow; elliptical. Tip: Acute. Base: Acute. Cross section: Concave.

Margin.—Entire; undulating.

Stipules.—None.

Color.—Upper surface (young and mature): Green Group 138B. Lower surface (young and mature): Green Group 138C. Veins (upper and lower surface): Grey-green.

Petioles: Length: 2-3 mm. Diameter: 1 mm. Color: Green Group 138D. Surface texture: Heavy pubescence.

Flowering: Naturally and continuously flowers in December—June in Israel. Colored White Group 155A. No fragrance. Year long flowering is achieved by growing the plant in a greenhouse covered with plastic (0.12 cm thick) with artificial daylight for 18 hours.

Growing conditions:

Winter irrigation.—3-4 m³ water with fertilizer per 1000 m² growing area per day.

Summer irrigation.—5-6 M³ water with fertilizer per1000 m² growing area per day.

Fertilizer.—150 ppm of 7-3-7.

I claim:

1. A new and distinct variety of Aerva javanica plant substantially as shown and described.
